(19) United States
(12) Patent Application Publication
(10) Pub. No.: US 2023/0019829 A1
(43) Pub. Date: Jan. 19, 2023

(54) FIRST RESPONSE APPARATUS, SYSTEM AND METHOD

(71) Applicant: MIME Technologies Ltd, Inverness (GB)

(72) Inventors: Elizabeth Anne Roberts, Inverness (GB); Alasdair James Mort, Inverness (GB)

(73) Assignee: MIME Technologies Ltd, Inverness (GB)

(21) Appl. No.: 17/787,903

(22) PCT Filed: Dec. 22, 2020
(86) PCT No.: PCT/EP2020/087696

§ 371 Date: Jun. 21, 2022

(30) Foreign Application Priority Data
Dec. 23, 2019 (GB) 1919141.0

(51) Int. Cl. G16H 40/63; A61B 5/00; G16H 15/00

(52) U.S. Cl. CPC G16H 40/63; A61B 5/0022; A61B 5/742; G16H 15/00

(57) ABSTRACT

A portable apparatus for use by first responders comprises at least one receiver configured to receive input from at least one medical sensor; a display device configured to display first response guidance to a user; user input device configured to receive input from the user; and processing circuitry configured to process the input from the at least one medical sensor and the input from the user; select the first response guidance to be displayed to the user via the display device, the selecting of the first response guidance comprising: estimating a status of the human subject based on the input from the at least one sensor, and selecting at least one item of first response guidance from a plurality of stored items of first response guidance based on the estimated status; wherein the first response guidance comprises at least one action to be performed by the user.

### FIELD

The present invention relates to an apparatus, system and method for first response to medical emergencies, for example medical emergencies occurring during flight.

### BACKGROUND

Every day, a large and increasing number of passengers travel by air. A small proportion of these passengers may experience medical emergencies during flight. A large airline carrier may deal with significant numbers of medical emergencies every year.

On some occasions, medical professionals may happen to be present on a flight and may be able to assist in a medical emergency. However, the standard protocol for responding to a medical emergency in flight is for the cabin crew to provide an initial response. The cabin crew are not medical professionals. In general, all cabin crew globally are trained with basic first aid skills. The cabin crew may often be faced with managing life-threatening situations. It may be difficult for cabin crew to remember the most appropriate passenger first aid guidance for specific first response presentations.

First aiders, for example cabin crew, may experience difficulties when using existing data capture, presentation and handover methods in a medical emergency. Many existing data capture systems are simple paper-based forms. Some airlines have data entry systems which record limited post-incident data. In some circumstances, vital information for handover to emergency systems may be lost. Forms may often be completed after an incident has occurred rather than while the incident is occurring. The filling of forms may therefore rely on the memory of the cabin crew. It is known that memory recall may be reduced in stressful circumstances.

Current products for first response are typically designed for professional use. First response products may include heavy, complex hardware. Such first response products may not be suitable for use in an airborne environment where weight is at a premium. Furthermore, some products may not work reliably after being stored in an aircraft for a long time. It is a feature of airborne medical emergencies that it is not possible to predict when an emergency will occur. Therefore, a first response system may be stored for many months before finally being used.

In some circumstances, products that are designed for professional use may require specialist knowledge for operation, which may make them unsuitable for use by a first aider who has had limited training. The operation of the products and/or the product interface may be complex which may make the product difficult to use by a non-specialist.

### SUMMARY

In a first aspect, there is provided an apparatus for use by first responders. The apparatus may be portable. The apparatus comprises at least one receiver configured to receive input from at least one medical sensor, wherein the input comprises or is representative of sensor data relating to a human subject. The apparatus further comprises a display device configured to display first response guidance to a user. The apparatus further comprises a user input device configured to receive input from the user. The apparatus further comprises processing circuitry configured to process the input from the at least one medical sensor and the input from the user and to select the first response guidance to be displayed to the user via the display device.

The selecting of the first response guidance may comprise estimating a status of the human subject based on the input from the at least one sensor, and selecting at least one item of first response guidance from a plurality of stored items of first response guidance, wherein the selecting is based on the estimated status. The first response guidance may comprise at least one action to be performed by the user in relation to the human subject.

The processing circuitry may be further configured to generate and output a medical handover report. The generating of the medical handover report may be performed automatically. The outputting of the medical handover report may be performed automatically. The generating and outputting of the medical handover report may be based on the input from the at least one medical sensor. The generating and outputting of the medical handover report may be based on the input from the user. The medical handover report may summarise medical information relating to the human subject.

The processing circuitry may be further configured to generate and output an incident report. The generating of the incident report may be performed automatically. The outputting of the incident report may be performed automatically. The generating and outputting of the incident report may be based on the input from the at least one medical sensor. The generating and outputting of the incident report may be based on the input from the user. The incident report may summarise medical information relating to the human subject. The incident report may comprise an airline incident report.

The apparatus may be an aviation apparatus for use by cabin crew.

The receiver, display device, user input device and processing circuitry may be housed within a single housing.

The at least one medical sensor may comprise at least one wireless sensor. The at least one receiver may be configured to receive the input from the at least one wireless sensor via a wireless connection. The wireless connection may comprise a Bluetooth® connection.

The at least one medical sensor may comprise at least one of an electrocardiography (ECG) sensor, a pulse oximeter, a heart rate monitor, a blood pressure sensor, a temperature sensor.

The first response guidance may comprise simplified guidance for use by first responders who are not medical professionals. The first response guidance may be generated in accordance with a standard first response protocol.

The estimating of the status of the human subject may be further based on the input from the user.

The least one action to be performed by the user in relation to the human subject may comprise administering a treatment to the human subject. The treatment may comprise oxygen. The least one action to be performed by the user in relation to the human subject may comprise inputting information relating to at least one sign or symptom of the human subject. The least one action to be performed by the user in relation to the human subject may comprise obtaining a manual measurement of a parameter relating to the human subject and inputting the manual measurement via the user input device. The least one action to be performed by the user in relation to the human subject may comprise performing a first response action. The first response action may comprise cardiopulmonary resuscitation. The first response action may comprise positioning the patient. The first response action may comprise lying the patient flat.

The processing circuitry may be further configured to generate an alarm. The generating of the alarm may be in dependence on the estimated status. The generating of the alarm may be in dependence on the input from the at least one medical sensor. The generating of the alarm may be in dependence on the input from the user. The generating of the alarm may be in dependence on a comparison between an input from the at least one medical sensor and at least one threshold. The generating of the alarm may be in response to the input from the at least one medical sensor exceeding the threshold. The threshold may distinguish input values that are considered to be normal from input values that are considered to be abnormal. The threshold may be set in accordance with first response guidelines. The threshold may be set in accordance with aviation guidelines. The threshold may be for use at altitude,

The generating of the medical handover report may comprise using natural language generation to generate a natural language description of information relating to the human subject. The generating of the medical handover report may comprise using natural language generation to generate a natural language description of actions performed in relation to the human subject. The generating of the incident report may comprise using natural language generation to generate a natural language description of information relating to the human subject. The generating of the incident report may comprise using natural language generation to generate a natural language description of actions performed in relation to the human subject.

The generating of the medical handover report may comprise collating input from the at least one sensor and input from the user. The generating of the medical handover report may comprise selecting or prioritising information from the collated input that is most relevant to the medical handover report. The generating of the incident report may comprise collating input from the at least one sensor and input from the user. The generating of the incident report may comprise selecting or prioritising information from the collated input that is most relevant to the incident report.

The handover report may be structured in accordance with a standard medical format. The standard medical format may be an ATMIST format (Age and other casualty details; Time of incident; Mechanism; Injuries sustained; Signs; Treatment and trends).

The processing circuitry may be configured to automatically timestamp the input from the at least one medical sensor to obtain timestamp data. The processing circuitry may be configured to automatically timestamp the input from the user to obtain timestamp data. The processing circuitry may be configured to use the timestamp data in the generating of the handover report.

The apparatus may be further configured to transmit data from the apparatus to an aircraft communications system. The aircraft communications system may comprise an in-flight Wi-Fi system. The transmitting of data may be performed in real time.

The transmitting of data may comprise receiving text input from the user of the apparatus and transmitting the text input. The processing circuitry may be further configured to receive and process a text reply, thereby providing text chat functionality.

The processing circuitry may be further configured to apply conditional formatting to the user interface. The applying of conditional formatting may be to highlight specific display items of the user interface in dependence on the estimated status of the human subject.

The user interface may be configured to receive input from the user via a one-touch input method. The processing circuitry may be configured to record one-touch input from the user. The processing circuitry may be configured to timestamp one-touch input from the user. The processing circuitry may be configured to record an input in response to a single touch from the user. The processing circuitry may be configured to timestamp an input in response to a single touch from the user.

There may be provided a system comprising an apparatus as claimed or described herein and at least one medical sensor.

In a second aspect, which may be provided independently, there is provided a method comprising: receiving, by at least one receiver, input from at least one medical sensor, wherein the input comprises or is representative of sensor data relating to a human subject; displaying, by a user interface, first response guidance to a user; receiving, by the user interface, input from the user; processing, by processing circuitry, the input from the at least one medical sensor and the input from the user; and selecting, by the processing circuitry, the first response guidance to be displayed to the user via the user interface. The selecting of the first response guidance may comprise estimating a status of the human subject based on the input from the at least one sensor. The selecting of the first response guidance may comprise selecting at least one item of first response guidance from a plurality of stored items of first response guidance, wherein the selecting is based on the estimated status. The first response guidance may comprise at least one action to be performed by the user in relation to the human subject.

There may be provided a computer program product configured to perform a method as claimed or described herein.

There may be provided an apparatus, system or method substantially as described herein with reference to the accompanying drawings.

Features in one aspect may be provided as features in any other aspect as appropriate. For example, features of a method may be provided as features of an apparatus and vice versa. Any feature or features in one aspect may be provided in combination with any suitable feature or features in any other aspect.

### BRIEF DESCRIPTION OF THE DRAWINGS

Various aspects of the invention will now be described by way of example only, and with reference to the accompanying drawings, of which:

FIG. 1 is a schematic diagram illustrating a first response system in accordance with an embodiment;

FIG. 2 is a flow chart illustrating in overview a method of an embodiment;

FIG. 3 is an illustration of a display screen of the first response system after launch;

FIG. 4 is an illustration of a further display screen showing signs and symptoms; and

FIG. 5 is a data flow diagram illustrating a flow of data in the system of FIG. 1;

FIG. 6 is a flow chart illustrating in overview a method of an embodiment in which a first response system is used for choking support; and

FIG. 7 is a flow chart illustrating in overview a method of an embodiment in which a first response system is used in a cardiac arrest scenario.

### DETAILED DESCRIPTION

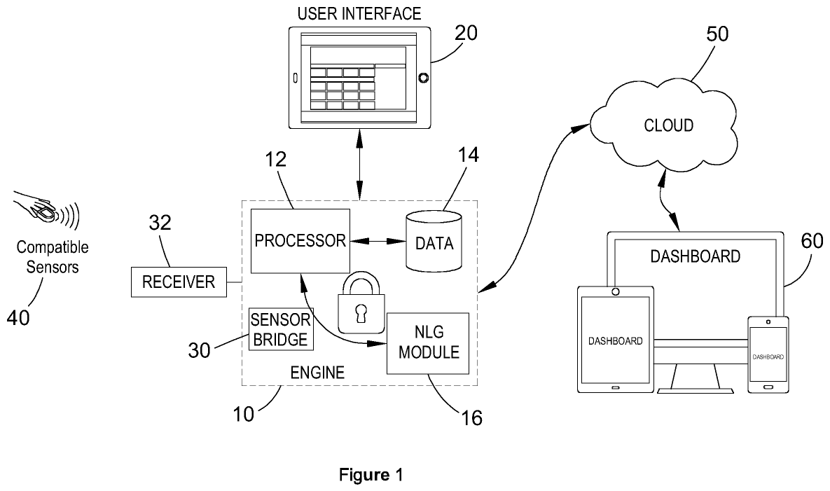

FIG. 1 is a schematic diagram illustrating a first response system in accordance with an embodiment. The first response system comprises a processing engine 10 comprising a processor 12, data store 14, and natural language generation (NLG) module 16. The processor 12 is configured to store data to the data store 14 and to receive stored data from the data store 14. The processor 12 is also configured to send data to, and receive data from, the NLG module 16. The NLG module 16 is configured to provide a natural language description of medical data as described below. The processing engine 10 is a secure unit. The processing engine 10 runs software that cannot be accessed or changed by a user of the first response system.

The first response system further comprises a user interface 20. The user interface 20 is configured to send data to, and receive data from, the processing engine 10. In the embodiment of FIG. 1, the processing engine 10 and user interface 20 each form part of the same computing apparatus, which in the embodiment of FIG. 1 is a tablet computing device. The processing engine 10 may comprise one or more processors of the tablet computing device. The user interface 20 is a touch screen of the tablet computing device.

In the present embodiment, the touch screen performs the function of a display device presenting a display to the user, and performs the function of a user input device receiving input from the user by the user operating the touch screen. References below to clicking an item displayed on the touch screen may comprise, for example, touching the item with a finger or stylus, or clicking with a mouse or touchpad or any other suitable input device. A simple one-touch input method may be used, in which clicking an item both records and timestamps the input from the user.

In some embodiments, the user interface 20 may comprise a display device (for example, a screen) for display to the user and a separate user input device (for example, a keyboard) for obtaining input from the user. In some embodiments, the display device forms part of a separate device from the processing engine 10 and/or the user input device. For example, the display device may be a screen that is separate from, but coupled to, a computing apparatus comprising the processing engine 10.

The tablet computing device is a portable device. The tablet computing device may be suitable for storage within an aircraft, in which storage space is typically limited. The tablet computing device houses both the processing engine 10 and the user interface 20 within a common housing. In other embodiments, any mobile device may be used that comprises the processing engine 10 and the user interface 20. Functionality of the processing engine 10 may be divided between two or more processors which may be situated in any appropriate device or devices.

The first response system further comprises a receiver 32 and a sensor bridge 30. In the present embodiment, the receiver 32 is a Bluetooth® receiver of the tablet computing device and comprises a Bluetooth® antenna. The receiver 32 is configured to receive wireless signals and convert the wireless signals into digital signals. The sensor bridge 30 is a software plug-in which is incorporated into the processing engine 10. In other embodiments, the sensor bridge 30 may comprise any suitable software or device.

The sensor bridge 30 is configured to receive data from the wireless sensors 40 wirelessly via Bluetooth® using the receiver 32. The sensor bridge 30 is further configured to pass the data from the wireless sensors 40 to the processor 12 or to at least one further part of the processing engine 10.

The sensor bridge 30 is further configured to receive data from the processor 12 or from at least one further part of the processing engine 10 and to pass the data to at least one of the wireless sensors 40 by instructing a transmitter (not shown) to transmit wireless signals to the at least one of the wireless sensors 40. The data may comprise instructions for at least one of the wireless sensors 40.

In other embodiments, any suitable wireless communication method may be used to pass data from the wireless sensors 40 to the processing engine 10 and/or from the processing engine 10 to the wireless sensors 40. In further embodiments, one or more of the sensors 40 may be connected via a wired connection. The receiver 32 may comprise any suitable hardware and/or software for receiving signals from the sensors 40.

The wireless sensors 40 are configured to measure physiological parameters of a human subject. In the description below, the human subject is referred to as a casualty. In other embodiments, the human subject may be any human subject, for example any casualty, patient, or other subject.

The wireless sensors 40 may comprise a wireless electrocardiography (ECG) sensor, for example a wireless 12 lead ECG patch. The wireless sensors 40 may comprise a wireless heart rate sensor. The wireless sensors 40 may comprise a wireless pulse oximeter. The wireless sensors 40 may comprise a wireless thermometer 40. The wireless sensors 40 may comprise a blood pressure cuff. In other embodiments, any wireless sensor or sensors may be used. The wireless sensors 40 may be configured to obtain any appropriate vital sign data. In further embodiments, one or more of the sensors may not be wireless and may be connected to the sensor bridge 30 and/or processing engine 10 by a wired connection. In the present embodiments, the wireless sensors 40 are low power devices.

The processing engine 10 is configured to send data to, and receive data from, a secure cloud-based service 50. The cloud-based service 50 comprises cloud-based processing and/or cloud-based storage. The cloud-based service 50 may be a software service that is hosted on any suitable computing apparatus or combination of computing apparatuses.

In the present embodiment, the processing engine 10 uses an in-flight Wi-Fi system of the aircraft (not shown) to connect to the aircraft's communication system (not shown). The aircraft's communication system sends data to the cloud-based service 50 using any suitable method, for example via ground-based mobile communications networks or via satellite communications.

In other embodiments, the processing engine 10 may not be connected to in-flight Wi-Fi. The processing engine may be connected to the cloud-based service 50 using any suitable connection, for example via internet, via a mobile communications network, or via a satellite connection.

A remotely-located dashboard device 60 is configured to receive data from, and send data to, the cloud-based service 50. For example, the dashboard device 60 may be located on the ground. The dashboard device 60 may be used by a ground-based medical provider. The dashboard device 60 may be located at an airline headquarters. The dashboard device 60 may comprise any suitable computing apparatus, for example a desktop computing device, laptop computing device, tablet computing device, or smartphone. The dashboard device 60 may connect to the cloud-based service 40 using any suitable connection, for example via internet, via a mobile communications network, or via a satellite connection.

The system of FIG. 1 is configured to run first aider mobile device software having interactive elements. In the embodiment of FIG. 1, the software is for use in the context of aviation, for example commercial aircraft and/or business jets. In other embodiments, the first response system and software may be used in any appropriate first response context. For example, the first response system and software may be used by professional or volunteer first responders in any urban or rural environment, in any form of transportation, or by mountain rescue. The first response system and software may be used in a maritime environment, in yachting, or in the offshore industry. The first response system and software may be used for any appropriate transmission of data regarding a first response incident.

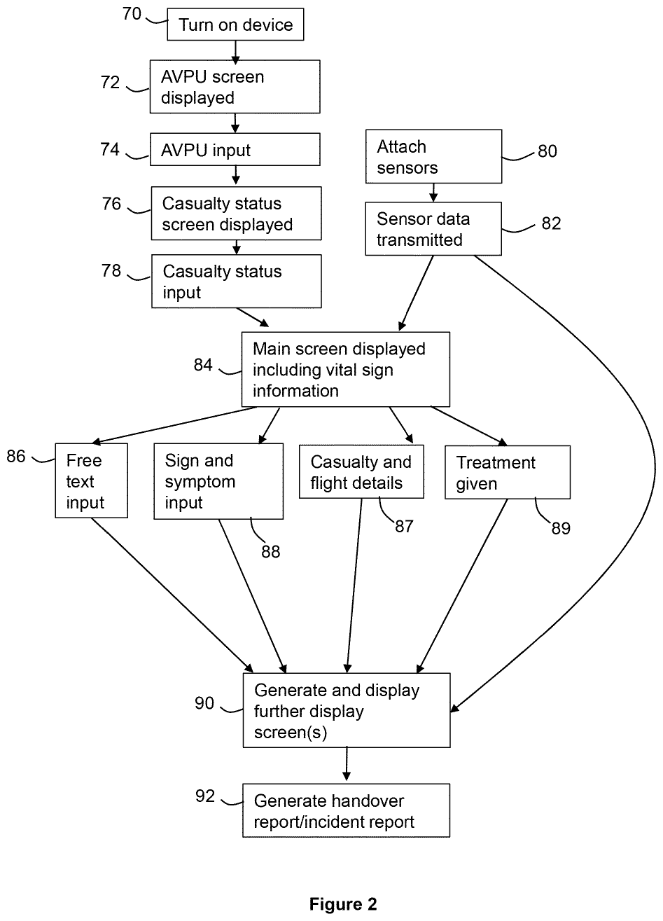

FIG. 2 is a flow chart illustrating in overview a method of using the first response system of FIG. 1. In the embodiment of FIG. 2, the first response system is used by an aircraft cabin crew member in response to a medical emergency concerning a passenger, other crew member, or any other human subject. The human subject may be referred to as a casualty. The human subject may have any known or unknown medical condition.

The flow chart of FIG. 2 is simplified and refers to a limited number of inputs, outputs and display screens. In other embodiments, any suitable combination of inputs, outputs and display screens may be used. The inputs, outputs and display screens may differ from those described in relation to FIG. 2.

At stage 70 of FIG. 2, the cabin crew member switches on the tablet computing device. The cabin crew member may select a display element (for example, an icon) on the screen of the tablet computing device to commence a first response activity.

At stage 72, the processor 12 instructs the user interface 20 (in this case, the screen of the tablet computing device) to display a screen display requesting the cabin crew member to check the casualty's AVPU status. Checking the casualty's AVPU status includes asking whether the casualty is alert; responds to verbal communication; responds to pain; or is unresponsive. In the present embodiment, the screen comprises four display elements.

At stage 74, the cabin crew member clicks on one of the four display elements (alert, verbal, pain, or unresponsive) to provide an AVPU input. The processor 12 receives the cabin crew member's input (alert, verbal, pain, or unresponsive). The processor 12 applies a timestamp to the input that is indicative of the time at which the input was provided, and stores data representative of the input and timestamp in the data store 14.

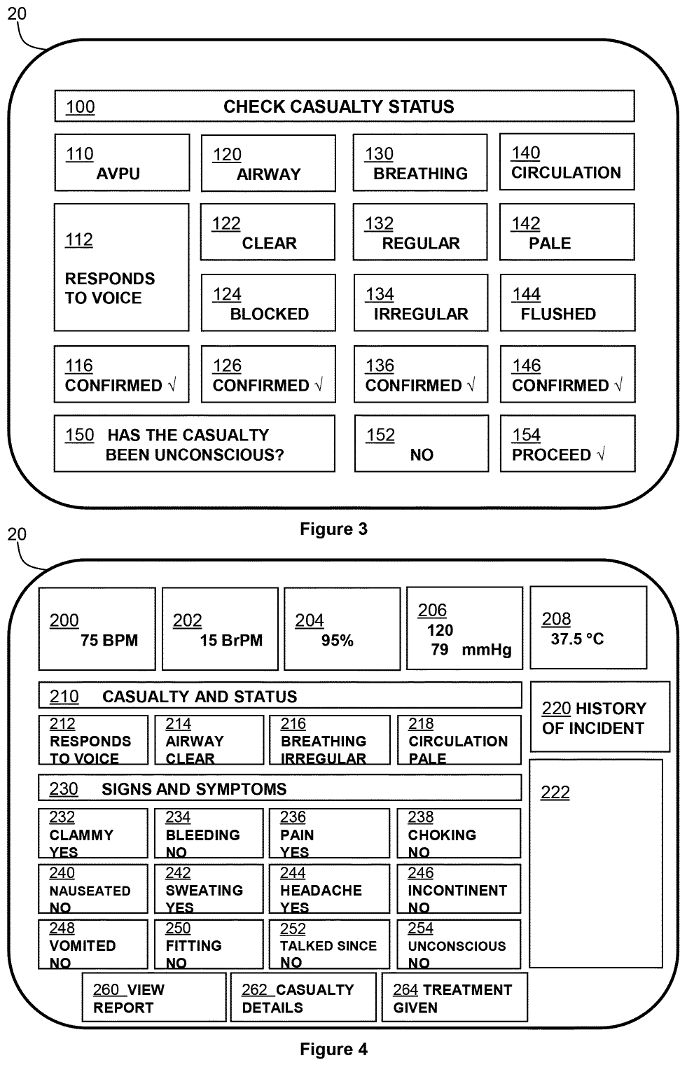

At stage 76, the processor 12 instructs the user interface 20 to display a casualty status display screen. An example of a casualty status display screen is illustrated in FIG. 3. In other embodiments, any suitable configuration of a display screen, or combination of display screens, may be used. The casualty status display screen asks the cabin crew member to provide various inputs. In other embodiments, the casualty display screen may include any appropriate first response guidance.

A title 100 of the display screen of FIG. 3 is ‘Check casualty status’. The display screen comprises four columns. A first column has a column heading 110 of ‘AVPU’.

A AVPU status display element 112 currently displays the text ‘Responds to voice’. An initial setting for the AVPU status display element 112 is set in accordance with the AVPU input at stage 74. Touching the display element 112 toggles the text of display element 112 between ‘alert’, ‘responds to voice’, ‘responds to pain’ and ‘unresponsive’.

A display element 116 requests the cabin crew member to confirm the setting for AVPU that is currently displayed by the display element 112. Once confirmed, the display element 116 displays the text ‘confirmed’ and a tick, as shown in FIG. 3.

Further columns of the display screen relate to ABC (Airway, Breathing and Circulation).

A second column of the display screen has a heading 120 of ‘Airway’. Two display elements ‘Clear’ 122 and ‘Blocked’ 124 are shown in the second column. In other embodiments, a further option of ‘Noisy’ may be provided.

One of the elements 122, 124 is highlighted, for example by colour. In the example shown, the ‘Clear’ element 122 is highlighted. The highlighted element is indicative of a status of the casualty. The cabin crew member may touch the unhighlighted element to switch the highlighting to that element.

A display element 126 requests the cabin crew member to confirm the setting for Airway that is currently displayed by the highlighting of the ‘Clear’ display element 122. Once confirmed, the display element 126 displays the text ‘confirmed’ and a tick, as shown in FIG. 3.

A third column of the display screen has a heading 130 of ‘Breathing’. Two display elements ‘Regular’ 132 and ‘Irregular’ 134 are shown in the second column. In the examiner shown, ‘Irregular’ 134 is highlighted. The cabin crew member may switch which of the elements is highlighted by touching the unhighlighted element as described above with reference to the second column.

A display element 136 requests the cabin crew member to confirm the setting for Breathing that is currently displayed by the highlighting of the ‘Irregular’ display element 134. Once confirmed, the display element 136 displays the text ‘confirmed’ and a tick, as shown in FIG. 3.

A fourth column of the display screen has a heading 140 of ‘Circulation’. Two display elements ‘Pale’ 142 and ‘Flushed’ 144 are shown in the second column. In the examiner shown, ‘Pale’ 142 is highlighted. The cabin crew member may switch which of the elements is highlighted by touching the unhighlighted element as described above with reference to the second column.

A display element 146 requests the cabin crew member to confirm the setting for Circulation that is currently displayed by the highlighting of the ‘Pale’ display element 142. Once confirmed, the display element 146 displays the text ‘confirmed’ and a tick, as shown in FIG. 3.

A further display screen element 150 asks the cabin crew member whether the casualty member has been unconscious. A response display element 152 currently displays ‘No’. The cabin crew may toggle the response display element 152 to ‘yes’ by touching the response display element 152.

Once all inputs have been provided by the cabin crew member, a display element 154 for ‘proceed’ is enabled. The cabin crew member may click on the ‘proceed’ display element 154 to progress to a next display screen. In the present embodiment, the next display screen is a main display screen as described below with reference to FIG. 4.

In response to pressing the ‘proceed’ element 154, the processor 12 commences logging an incident and starts to record data for that incident. All of the data that has been input up to the stage at which the ‘proceed’ element 154 is pressed is logged as one entry. The entry is given a timestamp corresponding to the time at which the ‘proceed’ element 154 is pressed. The data that has been input is stored in data store 14. The recording of data is performed automatically. In other embodiments, individual inputs may be timestamped at the time at which they are input.

At stage 80, the cabin crew member applies at least one wireless medical sensor 40 to the casualty. Stage 80 may be performed before, after, or simultaneously with any of stages 70 to 78. In some embodiments, the processor 12 instructs the user interface 20 to display one or more screen displays requesting the cabin crew member to apply at least one wireless sensor to the casualty. For example, the cabin crew member may be requested to apply a pulse oximeter to the casualty's finger and an ECG sensor to the casualty's body, if it safe and appropriate to do so. The screen display may include detailed guidance on how to apply the at least one wireless sensor. For example, the screen display may include correct electrode positions for the ECG sensor. In some embodiments, a screen display includes a display element for the cabin crew member to click when the or each sensor has been applied to the casualty. The processor 12 receives the cabin crew member's input indicating that the or each sensor has been applied, applies a timestamp to the input that is indicative of the time at which the input was provided, and stores data representative of the input and timestamp in the data store 14.

In other embodiments, the sensors attached may be any suitable sensors, for example non-wireless sensors. In some embodiments, the sensors used may not supply data directly to the processing engine 10. For example, the cabin crew member may make manual readings. In further embodiments, no sensors are applied to the casualty.

At stage 82, once the at least one wireless medical sensor 40 is operational, the processing engine receives sensor data from the at least one medical sensor 40 wirelessly via the sensor bridge 30. In other embodiments, sensor data may be received by a wired connection. The sensor data may comprise a continuous stream of sensor data. The continuous stream of sensor data may be representative of measurements taken at regular periodic intervals. The data comprises or is representative of at least one vital sign of the casualty. The processor 12 stores the sensor data in the data store 14. The processor 12 may also timestamp the sensor data, for example if the sensor data does not already include timing information.

At stage 84, the processor 12 instructs the user interface 20 to display a further display screen, which may be described as a main user interface display for monitoring signs and symptoms of the casualty. FIG. 4 is a schematic illustration of a main user interface display in accordance with an embodiment. The information shown on the main user interface display is obtained from the user's input in response to the AVPU and/or casualty status screens, and the sensor data transmitted from the at least one wireless sensor 40.

Values for five vital sign parameters are displayed on five display elements 200, 202, 204, 206, 208 across the top of the user interface 20. In the embodiment of FIG. 4, all of the vital sign data has been obtained from the at least one wireless sensor 40 applied to the casualty. In other embodiments, at least one item of vital sign data may be manually input by the cabin crew member.

Display element 200 shows heart rate in beats per minute (BPM). In the example shown in FIG. 4, the heart rate is 75 BPM. Display element 202 shows breathing rate in breaths per minute (BrPM). In the example shown in FIG. 4, the breathing rate is 15 BrPM. Display element 204 shows oxygen saturation as a percentage value. In the example shown in FIG. 4, the oxygen saturation is 95%. Display element 206 shows systolic and diastolic blood pressure as millimetres of mercury (mmHg). In the example shown in FIG. 4, the systolic blood pressure is 120 mmHg and the diastolic blood pressure is 79 mmHg. Display element 208 shows temperature in Celsius. In the example shown in FIG. 4, the temperature is 37.5° C.

FIG. 4 shows the display screen schematically and without colour. In practice, at least some of the vital signs display elements 200, 202, 204, 206, 208 may be highlighted in green, amber or red in dependence on the values for the vital signs. In the present embodiment, a traffic light format is used for heart rate 200, breathing rate 202, blood pressure 206 and temperature 208.

The traffic light format in green, amber or red may be used to indicate which values for vital signs are normal (green), are of concern (amber) or are of high concern (red). Each vital sign measurement that is shown in a traffic light format has a threshold between values shown as green and values shown as amber, and a threshold between values shown as amber and values shown as red. Thresholds for each of the vital signs may be set in dependence on aviation guidelines. Aviation guidelines may provide thresholds for vital signs at altitude. Thresholds for each of the vital signs may be defined by a customer, for example by an airline. In other embodiment, thresholds may be set in accordance with any suitable first response guidelines. The thresholds are stored in the data store 14 and applied to each item of vital sign data received by the processor 12 to determine a colour used when the vital sign data is displayed.

By using the traffic light format, the system may alert cabin crew if a vital sign measurement is higher or lower than a certain threshold. For example, the system may alert the cabin crew if oxygen saturation is below a given threshold. The system may alert the cabin crew if temperature is above a given threshold.

In some circumstances, it has been found that cabin crew may react with concern, for example by starting to panic, if colours of the vital signs display elements 200, 202, 204, 206, 208 change on the user interface 20 during an interaction with a patient. Therefore, in some embodiments, the vital signs display elements 200, 202, 204, 206, 208 are shown on the user interface 20 in a consistent colour, for example by consistently using black text on a white background.

Each vital sign display may display a current value for the vital sign measurement and/or an aggregated value, for example a rolling average of measurements obtained over a time interval. Each threshold may be applied to a current value and/or an aggregated value. In other embodiments, any suitable method of assessing whether a vital sign measurement is normal may be used. Any suitable method of visual indication (for example, colour, font, size, flashing or other visual effects) may be used to indicate abnormal vital sign measurements.

A heading 210 of ‘Casualty and Status’ is shown above four display elements 212, 214, 216, 218 which repeat the inputs shown on the previous screen of ‘Responds to Voice’ 212, ‘Airway clear’ 214, ‘Breathing irregular’ 216 and ‘Circulation pale’ 218. The cabin crew member may toggle the values of any of the four display elements 212, 214, 216, 218, for example to indicate if the AVPU, Airway, Breathing or Circulation status of the casualty changes. The processor 12 receives and timestamps any changes made to the display elements 212, 214, 216, 218.

The display screen of FIG. 4 further displays a heading 220 of ‘History of Incident’ and a free text box 222 below the heading 220. At stage 86 of FIG. 2, the cabin crew member enters free text describing the medical emergency into free text box 222. The cabin crew member may record any action or observation from the scene. The cabin crew member may record a narrative of a history of the medical emergency.

The processor 12 receives the free text input and stores the free text input to data store 14. The processor 12 may also timestamp the free text input. In other embodiments, the cabin crew member may not enter any free text input. In further embodiments, the cabin crew member may enter free text input at any appropriate time in the process and via any appropriate display screen.

At stage 88, the cabin crew member enters information relating to signs and symptoms. A heading 230 of ‘Signs and Symptoms’ is displayed above 12 display elements 232 to 254 that are representative of possible signs and symptoms. In the example shown in FIG. 4, each of the display elements 232 to 254 for the signs and symptoms displays a binary yes/no value. In some embodiments, at least some of the signs and symptoms may be initially displayed with a value of no. In some embodiments, initial values of at least some of the signs and symptoms may be based on a previous input, for example an AVPU input. In some circumstances, display element 254 may display a value of yes for unconscious. The display element 254 may display an indication of a length of time for which the casualty has been unconscious.

The user interface asks the cabin crew member to provide inputs regarding the signs and symptoms. The cabin crew member enters information regarding signs and symptoms by touching at least one of the sign or symptom display elements 232 to 254. Touching the display element for a sign or symptom may toggle the binary yes/no value for the sign or symptom.

In the embodiment of FIG. 4, the signs and symptoms shown are ‘Clammy’ 232, ‘Bleeding’ 234, ‘Pain’ 236, ‘Choking’ 238, ‘Nauseated’ 240, ‘Sweating’ 242, ‘Headache’ 244, ‘Incontinent’ 246, ‘Vomited’ 248, ‘Fitting’ 250, ‘Talked since’ 252, ‘Unconscious’ 254. In other embodiments, any suitable signs or symptoms may be included in the display screen.

Inputs regarding signs and symptoms are stored in data store 14. Inputs regarding signs and symptoms are timestamped with a time of the input.

In the embodiment of FIG. 4, signs and symptoms are recorded using a one-touch approach. The user needs only to touch the screen once to record a given sign or symptom and to timestamp the sign or symptom. The one-touch approach may be simple for the user. The one-touch approach may be a quick method of recording, which may assist in a time-critical scenario. In other embodiments, any suitable method of recording may be used.

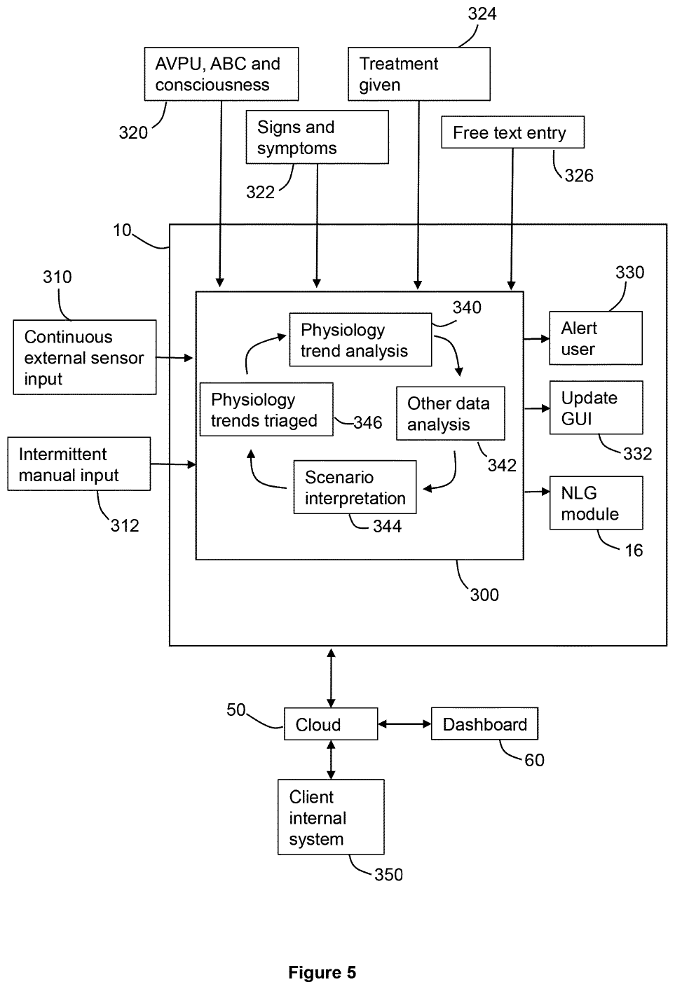

The display screen of FIG. 4 includes three further display elements 260, 262, 264 each of which, if touched, triggers the display of a respective further display screen. Display element 260 is labelled ‘View Report’ and takes the user to a report which may be generated as described below with reference to FIG. 5.

Display element 262 is labelled ‘Casualty details’ and takes the user to a display screen for input and/or display of details of the casualty, for example their name, age, gender, nationality, seat number, or other information. At stage 87, the cabin crew member touches display element 262 and enters casualty details, including flight details. The casualty details are stored in data store 14. The casualty details may be timestamped.

Display element 264 is labelled ‘Treatment given’ and takes the user to a display screen at which the user can record treatment given. The screen for recording treatment given may comprise a plurality of display elements that are representative of types of treatment that may be given. For example, the cabin crew member may touch a display element that is representative of the application of oxygen to indicate that oxygen has been applied to the casualty. Treatment given may be recorded using a one-touch approach. For example, the application of oxygen may be recorded and time stamped using a single touch. At stage 89, the cabin crew member touches display element 264 and inputs a treatment given. Details of treatment given are stored in data store 14. The details of treatment given are timestamped with a time at which they were input.

At stage 90, the processor 12 generates at least one further display screen (not shown) and instructs the user interface 20 to display the at least one further display screen to the cabin crew member. The at least one further display screen may be displayed in dependence on the input provides at stages 86, 87, 88 and/or 89. In other embodiments, at least one of the stages 86, 87, 88, 89 may be omitted. At least one further stage may be added. In some embodiments, no further display screen is displayed after the display screens of stages 86, 87, 88 and/or 89, and stage 90 is omitted. In some embodiments, the screen of stage 87 and/or the screen of stage 89 may be provided as a further display screen in dependence on stage 86 and/or stage 88.

Some specific inputs have been described above as being performed using a one-touch method of recording. In other embodiments, a one-touch method may be used for any suitable input by a user. For example, a cabin crew member may only have to press a display element representative of any sign, symptom or treatment path once for the system to record and time stamp the cabin crew member's input.

Although FIGS. 2 to 4 show a specific arrangement and ordering of screens, in other embodiments any suitable arrangement of display elements and/or display screens may be used. The software may provide one or more Help screens which provide tailored guidance to the user. In some embodiments, a user may touch a ‘Help’ display element to obtain additional help and guidance at any point while using the system.

The display screens may provide any appropriate first response guidance to the cabin crew member. The first response guidance may correspond to first aid training received by the cabin crew member. The first response guidance may comprise a request for input. The requested input may comprise, for example, input regarding AVPU; information regarding signs and/or symptoms; information regarding the casualty; or manual sensor input. The first response instructions may comprise an action to be taken by the cabin crew member. For example, the cabin crew member may be asked to consider performing a first aid action such as performing CPR (cardiopulmonary resuscitation), if safe and appropriate to do so. The cabin crew member may be directed to a possible form of treatment, for example to an item in the first aid kit.

Stage 92 of FIG. 2 is performed at the end of an episode of use of the first response system, for example when the casualty is being handed over to a medical professional on landing. At stage 92, the processor 12 instructs the NLG module 16 to generate a medical handover report. In the present embodiment, the processor 12 also instructs the NLG module to generate an incident report, for example for the airline. The incident report may be the same as the medical handover report. The incident report may be different from the medical handover report, for example including different information or using a different format. The incident report may also be referred to as an audit report.

The medical handover report may be tailored to specific first response scenarios. In the present embodiment, the medical handover report is developed in accordance with an ATMIST format. The ATMIST format is a standard pre-hospital care format. The ATMIST format orders handover information by Age and other casualty details; Time of incident; Mechanism; Injuries sustained; Signs; Treatment and trends.

In other embodiments, any suitable reporting format may be used.

In the present embodiment, the NLG module 16 is configured to translate variable names and corresponding variables that are entered in a non-English language into an English equivalent. The translation into English may allow easier understanding and handover and/or audit reports for airline use.

In some embodiments, the handover report comprises a natural language summary of inputs provided from the sensors and/or cabin crew member. The handover report may include details of vital sign data, for example the occurrence and timing of changes in vital sign data. Any suitable method of displaying vital sign data may be used. For example, vital sign data over time may be presented in one or more graphs. Text may be generated to accompany a graph, for example text describing a trend occurring in the data of a graph.

Each sign or symptom may have a corresponding text description which is included in the handover report if the sign or symptom is present. A time at which the sign or symptom was recorded may also be included.

The handover report may include information on actions performed by a user. A natural language description may be provided comprising each action and a time at which the action was performed.

The handover report may collate information that has been input from multiple sources, for example multiple sensors. The handover report may collate information that has been input over a period of time, for example over minutes or hours.

In some embodiments, the NLG module 16 determines which information is to be included and/or prioritised in the handover report. For example, abnormal vital sign information may be prioritised over normal vital sign information. The handover report may highlight any apparent errors in the data, and/or data that haven't been recorded.

The handover report may provide an indication of which information is most important and/or urgent. For example, sections of the handover report may be highlighted. An ordering of the handover report may be in dependence on importance and/or urgency.

The use of NLG enables reports to be automatically produced in text format. The cabin crew do not have to write any incident data. The report is automatically timestamped with any actions and observations. NLG reporting may improve information gathering and reduce the time taken to collect and relay relevant information during emergency and non-emergency scenarios. Accuracy of reporting may be improved. Completeness of reporting may be improved. The handover report may be more suited to use by subsequent medical professionals than a handover report made by a cabin crew member, for example made verbally by a cabin crew member, or by a form filled in manually by a cabin crew member. An incident report may be prepared in a standard format. The format of the incident report may automatically be compliant with airline requirements or other industry requirements.

Data may also be provided to a ground-based medical provider using the dashboard 60. The data may be provided via the cloud-based system 50. The data may include live data. The provision of live data may depend upon the availability of the plane's communications network and/or the latency of the plane's communications network. The dashboard 60 may show inputs that have been made to the user interface 20. The data provided to the ground-based medical provider may include the handover report.

The data provided to the ground-based medical provider may include any items of data that are displayed to, or provided by, a cabin crew as described above. Data provided to the ground-based medical provider may show the ground-based medical provider the same information that is being shown to the cabin crew member.

In some embodiments, vital signs data is shown on the dashboard 60 using a traffic light format as described above. The traffic light format in green, amber or red may be used to indicate which values for vital signs are normal (green), are of concern (amber) or are of high concern (red). Threshold values may be used as described above. By using the traffic light format, the system may alert the ground-based medical provider if a vital sign measurement is higher or lower than a certain threshold. By colour coding the physiological information that displays on the dashboard 60 used by the ground-based medical provider, the ground-based medical provider may be alerted straight away to updates. The ground-based medical provider may review updates in vital sign data and may recommend appropriate action to the pilot or crew.

In some embodiments, the vital sign information is displayed to the cabin crew member in a consistent format that does not use traffic light colouring or other highlighting, while the same information when displayed to the ground-based medical provider is displayed with traffic light colouring or other appropriate highlighting, to assist the ground-based medical provider in assessing changes.

A ground-based medical provider may be a doctor or other clinician. The ground-based medical provider may be situated in any location that is remote from the aircraft. The ground-based medical provider may provide an expert opinion on the first response event. For example, the ground-based medical provider may assist a pilot of the aircraft to decide whether to divert the aircraft, or whether to proceed to the aircraft's original destination. The information received via the dashboard 60 may assist the ground-based medical provider in providing an expert opinion. A combination of data, actions and observations together may provide more information to make a more informed decision on diversion.

The providing of data to a ground-based medical provider, or other remote user, is described in more detail below with reference to FIG. 5.

FIG. 5 is a flow chart illustrating in overview a flow of data into and out of the processing engine 10 in accordance with an embodiment. Any of the data described in FIG. 4 may be stored in and/or retrieved from data store 14.

At stage 310, the processing engine 10 receives continuous external sensor input from at least one wireless medical sensor. The continuous external sensor input may comprise a stream of sensor data comprising data from measurements taken at regular periodic intervals. The measurements may be obtained automatically by the at least one wireless medical sensor. The sensor data may comprise, for example, heart rate data and/or oxygen saturation data.

At stage 312, the processing engine 10 receives intermittent manual input of sensor measurement data. For example, a cabin crew member may use a medical sensor to acquire a measurement of a vital sign. The cabin crew member may manually input the vital sign measurement to the processing engine 10 by entering the vital sign measurement via the user interface 20. In some circumstances, manual input may be used if there is no connectivity between the medical sensor 40 and the processing engine 10. The data that is manually input may comprise, for example, blood pressure data and/or temperature data.

In other embodiments, any method of providing sensor input may be used, which may or may not be continuous.

At stage 320, the processing engine 10 receives information regarding AVPU, ABC and consciousness which is input by the cabin crew member via the user interface 20. For example, AVPU, ABC and consciousness information may be input via the display screen shown in FIG. 3. Information input via the user interface 20 may be timestamped by the processor 12. The AVPU, ABC and consciousness information may include, for example, an indication that the casualty is alert; an indication that the casualty responds to pain; or an indication that the casualty has been unconscious for x minutes.

At stage 322, the processing engine 10 receives information regarding signs and symptoms that is input by the cabin crew member via the user interface 20. For example, sign and symptom information may be input via the display screen shown in FIG. 4 and may be timestamped by the processor 12. The sign and symptom information may comprise, for example, information on nausea, bleeding or choking.

At stage 324, the processing engine 10 receives information regarding treatment given that is input by the cabin crew member via the user interface 20. For example, information on treatment given may be input on a treatment display screen. Inputs on treatment given may be timestamped by the processor 12. The treatment given may comprise, for example, applying oxygen or moving the casualty.

At stage 326, the processing engine 10 receives free text information via free text entry by the cabin crew member using the user interface 20. For example, free text information may be input through a free text box 222 such as that shown in FIG. 4. Free text information may be timestamped by the processor 12. The free text information may include, for example, additional casualty details.

The sensor input, manual vital sign input, AVPU, ABC and consciousness information, sign and symptom information, treatment given information, and free text information are supplied to a decision support module 300. Other embodiments may have any suitable combinations of inputs. In some embodiments, the decision support module 300 is an artificial intelligence decision support module which deploys artificial intelligence to provide decision support.

The decision support module 300 provides a cycle of analysis based on ongoing inputs. Incoming values are analysed in real time and passed through a scenario interpretation module.

Decision support processes for first aid may help the cabin crew and alert them to potential first response scenarios. Decision support processes may be provided in addition to general algorithm-based first aid steps. Decision support processes are implemented around the information and decision making made available to the cabin crew.

At stage 340, the decision support module 300 performs an analysis of one or more physiological trends. For example, the trends may be based on trends in sensor input (for example, blood pressure or temperature) and/or on trends in other inputs (for example, a change from unconsciousness to consciousness).

At stage 342, the decision support module 300 performs other data analysis, for example data analysis on data that does not relate to physiological trends. The data analysis of stage 342 may make use of outputs of the physiological trend analysis of stage 340.

At stage 344, the decision support module 300 performs a scenario interpretation. The decision support module may estimate a status of the casualty. The estimating of the status of the casualty may be at least partially based on an output of the data analysis of stage 342.

At stage 346, the decision support module 300 triages physiological trends. The triage of physiological trends is based at least in part on the estimated status of stage 344.

The analysis then returns to physiological trend analysis at stage 340. The physiological trend analysis of stage 340 may be at least partially based on an output of the triage of physiological trends at stage 344.

In the present embodiment, the decision support module 300 estimates a status of the casualty. The status may comprise a clinical scenario. The status may comprise, for example, an abnormal reading for at least one vital sign. The status may be dependent on at least one sign or symptom. In some embodiments, the status is estimated using a fixed algorithm, for example a first aid algorithm. In other embodiments, the status is estimated using more complex decision support, for example using artificial intelligence.

Medical conditions that occur most frequently in the aviation condition may include, for example, syncope, respiratory conditions, choking, vomiting, chest pain, palpitations, cardiac arrest, seizures, abdominal pain, and psychiatric complaints. Specific examples of decision support are described below.

The output of the decision support does not comprise a diagnosis as such. Instead, the output may comprise an estimate of a status which may relate, for example, to a particular medical condition (for example, respiratory) or to a particular vital sign (for example, blood pressure).

The decision support module 300 may produce external outputs at any point in the cycle of stages 340, 342, 344 and 346.

A first form 330 of external output is to provide an alert to a user, for example a cabin crew member or to a ground based medical provider. The alert may comprise an audible and/or visual alarm, for example a loud sound and/or flashing light. The alert may comprise a text display, for example a written warning displayed on the user interface 20. The alert may comprise a change in an existing display screen, for example by turning one or more of the display elements red.

In some embodiments, no alarm may be provided to the cabin crew member but an alarm may be provided to a ground based medical provider. The ground based medical provider may ask further questions of the cabin crew based on the alarm, for example to determine whether the alarm may have been caused by a sensor malfunctioning or becoming detached. In further embodiments, no alarm is issued and external output 330 is omitted.

The user may be alerted to perform various actions. For example, if blood saturation drops and oxygen has not yet been applied, the system may alert the cabin crew member to apply oxygen to the casualty.

A second form 332 of external output is an update to the user interface 20, which may also be referred to as a graphical user interface or GUI. The update to the user interface 20 may comprise a change in one or more of the display elements. The update to the user interface 20 may comprise a change in a displayed parameter. The update to the user interface 20 may comprise a change from a display screen to a different display screen. The update to the user interface may comprise the display of one or more items of guidance to the user.

In some embodiments, the change in user interface 20 comprises the use of conditional formatting to highlight issues around vital signs and/or symptoms. A visual characteristic of any appropriate display element of the display screen may be changed to highlight the information shown in that display element. For example, a colour, font or size may be changed and/or the display element may flash or otherwise change over time. Formatting may be changed in dependence on a value for a sign of symptom or in dependence on a particular clinical scenario.

Information provided to the cabin crew member via the user interface 20 asks the cabin crew member to consider performing initial first aid treatment responses, if safe and appropriate to do so. The information is based on the estimated status. Initial first aid treatment responses may comprise, for example, considering starting CPR, considering oxygen, or considering lying the casualty flat.

In some embodiments, information provided to the cabin crew member via the user interface 20 comprises information to aid further questioning by or of the cabin crew member. In some embodiments, information provided to the cabin crew member via the user interface 20 comprises information to assist in navigating an inflight medical kit to find a relevant medication for an emerging issue. First aid decision support may be derived from a combination of sensor data and actions and/or observations from first aid.

A third form of external output is an output to the natural language generation module 16. Information may be provided to the NLG module 16 to enable the NLG module to generate a handover report as described above.

In embodiments, two or more of the different forms of external output may be combined. The external outputs may be provided in any order.

Data is also transmitted from the processing engine 10 to a cloud-based service 50. The data may be live streamed to the cloud-based service. The data transmitted to the cloud-based service 50 may comprise any of the information that is received by the processing engine 10 at stages 310, 312, 320, 322, 324, or 326. Data is transmitted from the cloud-based service 50 to a dashboard 60 as described above with relation to FIG. 1. Data may alternatively or additionally be transmitted to a client internal system 350, for example an airline system. Data may be transmitted via a REST API.

In-flight data is streamed to the cloud-based service 50. In-flight data that has been streamed to the cloud-based service 50 can be viewed via a dashboard 60. The dashboard 60 may be used by airline ground-based medical providers. The ground-based medical providers may support the captain with making a decision of whether or when to divert the aircraft. If the airline does not have a ground-based medical provider, the dashboard 60 may be provided in the airline headquarters. A medical representative at the airline headquarters can view the data in real time. In other embodiments, data may be sent to any suitable interested party using any suitable format and transmission method.

In the present embodiment, the transmission of data to the dashboard 60 via the cloud-based service 50 includes the ability to hold a live two-way text chat with a ground-based medical provider. Chat functionality may be used in a particular sequence of events, for example during an inflight medical incident. The cabin crew member inputs text, for example a question, into a chat text box. The chat text box forms part of any suitable display screen on the user interface 20. The text that is input by the cabin crew member is transmitted to the cloud-based service 50 and from the cloud-based service 50 to the dashboard 60. A ground-based medical provider views the dashboard 60 and reads the cabin crew member's text input. The ground-based medical provider types in a response to the cabin crew member, for example an answer to the cabin crew member's question. The ground-based medical provider's response is transmitted to the cloud-based service 50 and from the cloud-based service 50 to the processing engine 10. The ground-based medical provider's response is displayed to the cabin crew member in the chat text box.

The use of chat functionality may allow cabin crew to speak in real time to ground based medical providers without leaving the passenger's side.

In some known systems, if a cabin crew member wished to speak to a ground-based medical provider about an ongoing medical incident, the cabin crew member would be required to leave the casualty's side to communicate with the ground-based medical provider via a telephone of the aircraft. It is undesirable to leave the casualty unattended while communicating with the ground-based provider as may be required if the aircraft's telephone is used. In some systems, the cabin crew member may speak to the ground-based medical provider via another voice connection that does not require the cabin crew member to leave the casualty. However, communication via voice connection may be subject to interference, for example interference from the aircraft. Communication may be degraded by noise and/or vibration from the aircraft. Voice communication may be challenging if there are language and/or access barriers between the cabin crew member and the ground-based medical provider. For example, voice communication may be misinterpreted. Communication by chat may provide a clearer form of communication that is less subject to interference. Communication by chat may require significantly less telecommunications bandwidth than voice. Chat may be a more resilient form of communication than voice communication, for example in locations with challenging signal availability. Chat communication may provide ground based medical providers with an easier way to review and recommend. Furthermore, communication by chat may provide better records of conversations that would be provided in the case of voice communication.

By using the first response system, a first responder with limited training such as a cabin crew member may be supported in a first response scenario. First aid algorithms provide tailored guidance to the cabin crew member at appropriate times. Guidance may be provided using software help screens.

Data is presented to the user in a very simple user interface. Data can also be entered manually. For example, manual data entry may be used if the crew do not have Bluetooth® connectivity to the medical monitoring equipment. Vital signs data is displayed on the main interface, which is illustrated in FIG. 4. The user interface of FIG. 4 displays signs and symptoms in an easy to read format.

Data collected using the first response system may be transmitted to a remote location (for example, to a ground based medical provider). In some circumstances, the data transmitted may be acted on immediately. Data may be stored locally in the data store 14 or in any appropriate data store. Data may be stored in the cloud, for example using the cloud-based service 50. Stored data may be used for future reporting. Stored data may be reviewed at any suitable future date.

The processes provided by the first response system may not be intended for diagnostic purposes. Instead, the processes may be an aid to initial first aid steps, in line with airline cabin crew training protocols. The processes may assist the cabin crew and a ground-based service to manage an event. Decision support may also help clinicians on the ground to review and recommend most appropriate actions. A combination of data and decision support may help the pilots and a clinician on the ground to decide on whether to divert the aircraft or not.

Some specific examples of inputs and actions are described below.

An input may be provided for Airway as described above. The method of Airway input may ask if the casualty's airway is open and unobstructed. If the input is Yes, it may be the case that no action is suggested based on the Airway input alone. In the input is No, guidance may be provided which asks the cabin crew member to consider performing an action to open the casualty's airway. For example, the cabin crew member may be asked to consider a chin lift or jaw thrust, and if the airway remains obstructed to consider physical obstruction. The cabin crew member may be asked to consider inserting an airway.

An input may be provided for Breathing (Type) as described above. If the input is No, the cabin crew member may be asked to consider CPR. If the input is Yes, it may be the case that no action is suggested based on the Breathing input alone. The breathing may be regular and normal.

In some embodiments, one possible input for Breathing (Type) is Noisy. If the input is Noisy, the cabin crew member may be asked to consider obstruction of the airway. Further breathing types may also be input. If the breathing is quiet and/or laboured and/or intermittent, the cabin crew member may be asked to consider obstruction and/or CPR.

A sensor input may comprise respiratory rate in breaths per minute. A breathing rate of 12 to 20 breaths per minute may be considered to be normal for an adult. For example, a traffic light threshold may be set such that a breathing rate of 12 to 20 breaths per minute is highlighted in green. A breathing rate of 0 to 11 breaths per minute may be considered to be abnormal. If the breathing rate is between 0 and 11 breaths per minute, the cabin crew member may be asked to consider CPR. A breathing rate of 25 or more may be considered to be abnormal. In the case of a school age child, a normal range may be between 18 and 30 breaths per minute.

A sensor input may comprise pulse rate in beats per minute. A normal range for pulse rate in beats per minute may be between 60 and 80 beats per minute.

A sensor input may comprise oxygen saturation as a percentage. It is known that oxygen saturations at altitude may usually be up to 5% lower than on the ground. Therefore, oxygen saturations may be used mainly for information and/or for obtaining trends.

If oxygen drops below a certain aviation specific threshold set by the airline, the decision support module 300 may prompt the cabin crew to consider applying oxygen. The decision support module may only prompt the cabin crew to consider applying oxygen if oxygen hasn't already been applied, and there are no contra-indications (for example, the casualty having COPD).

A sensor input may comprise temperature, for example in degrees Celsius. A temperature of between 35.8° C. and 37° C. may be considered normal. A temperature of 38° C. or above may be considered abnormal. If the temperature is 38° C. or above, the cabin crew member may be asked to complete a checklist of questions to ascertain whether the casualty has a communicable disease. The checklist of questions may be in accordance with standard cabin crew guidance concerning communicable diseases.

AVPU, ECG and actions taken may also be recorded.

Some specific examples of decision support scenarios are described below. The processing engine, for example the decision support module 300, may be configured to perform any one or more of the decision support scenarios below. In other embodiments, the processing engine 10, for example the decision support module 300, may be configured to perform any suitable decision support process, which may not comprise one of the decision support examples described below. In other embodiments, individual features or actions of the decision support processes may be different from those described below.

Decision support may be used for the scenario of a simple faint or syncope. Decision support may be used in a choking scenario. Decision support may be used in a chest pain scenario. The chest pain may be a heart attack. The chest pain may be a stable chest pain. The chest pain may be a non-cardiac chest pain. Decision support may be used in a choking scenario. Decision support may be used in the case of a potential communicable disease. Decision support may be used in an allergy scenario.

In an example of a faint or syncopal episode, inputs are obtained at a first time which may be labelled as a time of 0 minutes. An AVPU input indicates that the casualty is unconscious. The airway inputs indicates that the airway is open. The breathing input indicates that the casualty's breathing is normal and regular. The respiratory rate is 18 breaths per minute. The pulse rate is 84 beats per minute. The oxygen saturation is 94%. The temperature is 37.2° C.

The decision support module 300 processes the inputs and provides guidance to the cabin crew member. The guidance may ask the cabin crew member to move the passenger to the floor or across seats with their legs up. The guidance may suggest a possibility of faint or syncope, for example by suggesting that the cabin crew member consult information on faint or syncope in an internal first aid manual. The cabin crew member may provide an input, for example a one-touch input or a text input, indicating that the passenger has been moved or providing other input.

At a time of 1 minute, the AVPU input is updated to indicate that the casualty is alert. Inputs indicate that the airway is clear; breathing is normal and regular; breathing rate is 20 breaths per minute; pulse rate is 90 beats per minute; oxygen saturation is 93%; and temperature is 37.2° C.

At a time of 4 minutes, the pulse rate is 88 beats per minute; oxygen saturation is 94%; and temperature is 37.2° C. A text input indicates that the casualty has been reassured. At a time of 5 minutes, the casualty is alert; airway is clear; breathing is normal and regular; and the breathing rate is 18 breaths per minute.

The incident may be considered to be complete when the casualty has returned to a normal status. The decision support module 300 may provide an output indicating that the casualty's status may be considered to be normal. A handover report and/or incident report may be generated.

An example of a choking scenario is now described. At a first time of 0 minutes, the casualty is alert but unable to speak. In some embodiments, alert but unable to speak is provided as an input option, for example a one-touch input. In some embodiments, it is input only that the casualty is alert. In some embodiments, the information that the casualty is unable to speak is provided as a text input.

At the time of 0 minutes, an input of ‘No’ is recorded for Airway. The casualty points to their throat. The casualty has increasing panic. There is a history of being blocked with food. Information about the casualty's behaviour and/or history may be input as a one-touch input or text input, or may not be input.

At the time of 0 minutes, the casualty's breathing is input as Quiet. The breathing rate is between 0 and 12 breaths per minute; pulse rate is 120 beats per minute; oxygen saturation is 94%, and temperature is 37° C.

The decision support module 300 receives the input and outputs guidance that the cabin crew member consider back blows and, if unsuccessful, abdominal thrusts. The cabin crew member (or another cabin crew member) may input that back blows and abdominal thrusts have been performed.

At a time of 1 minute, inputs indicate that the casualty is alert; the airway is not open; breathing is quiet; respiratory rate is between 0 and 6 breaths per minute; pulse rate is 130 beats per minute; and oxygen saturation is 88%. The decision support module 300 outputs guidance that the cabin crew member consider continuing cycles of back blows and abdominal thrusts. The cabin crew member may input that back blows and abdominal thrusts have been performed.

At a time of 2 minutes, inputs indicate that the casualty is alert.

At a time of 3 minutes, inputs indicate that the casualty is alert and the airway is now open. Respiratory rate is 20 breaths per minute; pulse rate is 90 beats per minute; oxygen saturation is 94%; temperature is 37.5° C.

At a time of 4 minutes, inputs indicate that the casualty is alert.

At a time of 10 minutes, guidance is given that if abdominal thrusts have been used, the passenger must seek medical attention on the ground. A ground based medical provider may consider data provided by the first response system. In consideration of the data provided by the first response system, the ground based medical provider may advise that the flight does not need to be diverted. A handover report and/or incident report may be generated.

An example of a cardiac chest pain scenario is now described. At a time of 0 minutes, inputs indicate that the casualty is alert; airway is clear; breathing is normal; and breathing rate is 20 breaths per minute. Pulse rate is 60 beats per minute but may be dropping. Oxygen saturation is 92% and temperature is 37° C. At least some of the casualty's history may be input as text input. The casualty is panicky, very frightened, complains of chest pain, has a history of heart problems, and is feeling nauseated.

At a time of 1 minute, the casualty is unconscious. The cabin crew member inputs that the airway is not clear, and is asked to consider head tilt/chin lift or jaw thrust. The cabin crew member inputs that breathing is noisy, and is asked to consider head tilt/chin lift or jaw thrust.

The respiratory rate is 6 breaths per minute and the cabin crew member is asked to consider CPR. Pulse rate is 15 beats per minute. Oxygen saturation is 86% and temperature is 37° C.

The decision support module 300 analyses inputs and provides guidance to call for help; call for AED (automated external defibrillator); consider head tilt/chin lift or jaw thrust; and consider CPR compressions. The cabin crew member (or another cabin crew member) may provide input indicating that the AED has been applied and/or that any of the other actions has been performed.

At a time of 2 minutes, inputs indicate that the casualty is unconscious; the airway is not clear; breathing is quiet; and respiratory rate is 0.

At a time of 3 minutes, inputs indicate that the casualty is unconscious. At a time of 4 minutes, inputs indicate that the casualty is unconscious.

At a time of 10 minutes, inputs indicate that the casualty responds to voice. The airway is open and breathing is noisy. The cabin crew member is asked to maintain the airway. The respiratory rate is 12 breaths per minute; pulse rate is 60 beats per minute; oxygen saturation is 92% and temperature is 36.5° C.

The decision support module 300 issues guidance advising the cabin crew member that it appears that there has been a return of circulation and breathing. The cabin crew member is asked to continue to monitor and keep the passenger warm with a blanket, reassuring where possible.

An example of a communicable disease scenario is now described. At a time of 0 minutes, inputs indicate that the casualty is alert. The airway is clear and breathing is normal. The respiratory rate is 26 breaths per minute; pulse rate is 130 beats per minute; oxygen saturation is 94% and temperature is 39.2° C.

The decision support module 300 processes the inputs and asks the cabin crew member to consider possible communicable disease and follow a question list and advice.

At 1 minute and 2 minutes, inputs indicate that the casualty is alert.

In any of the above scenarios, a ground based medical provider may review inputs and make recommendations to the cabin crew member and/or to the pilots.

In summary, the system comprises software running on a mobile device which links to wireless, Bluetooth® low energy vital sign medical sensors. The system begins by guiding cabin crew through the process of first aid according to national and international resuscitation protocols. It asks if the passenger is alert, responds to voice, responds to pain or is unconscious (AVPU). First aid algorithms provide tailored guidance at appropriate times, using software Help screens. The system links using Bluetooth® wireless low energy sensors (such as a wireless 12 lead ECG patch) and data are presented in a very simple user interface. Data can also be entered manually, for example if the crew do not have Bluetooth® connections to medical monitoring equipment. Vital sign data is displayed on a main interface. Data are logged and time stamped on the mobile device. The processing engine 10 takes in all information gathered from the medical sensors and the data inputted manually by one or more users. For example, data inputting manually by users may comprise their actions and observations on scene. The processing engine 10 produces a handover report to emergency services and/or for airline audit. The handover report is produced using natural language generation code, for example using an ATMIST format. In-flight data is streamed to the cloud-based service 50 and from the cloud-based service 50 to the dashboard 60, for example for use by ground based medical providers.

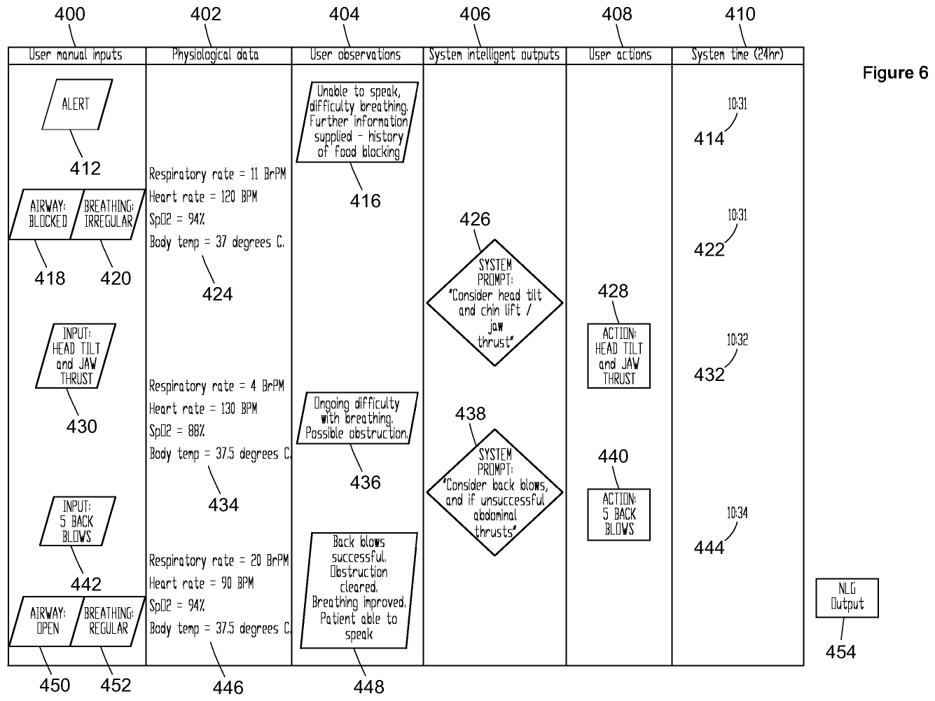

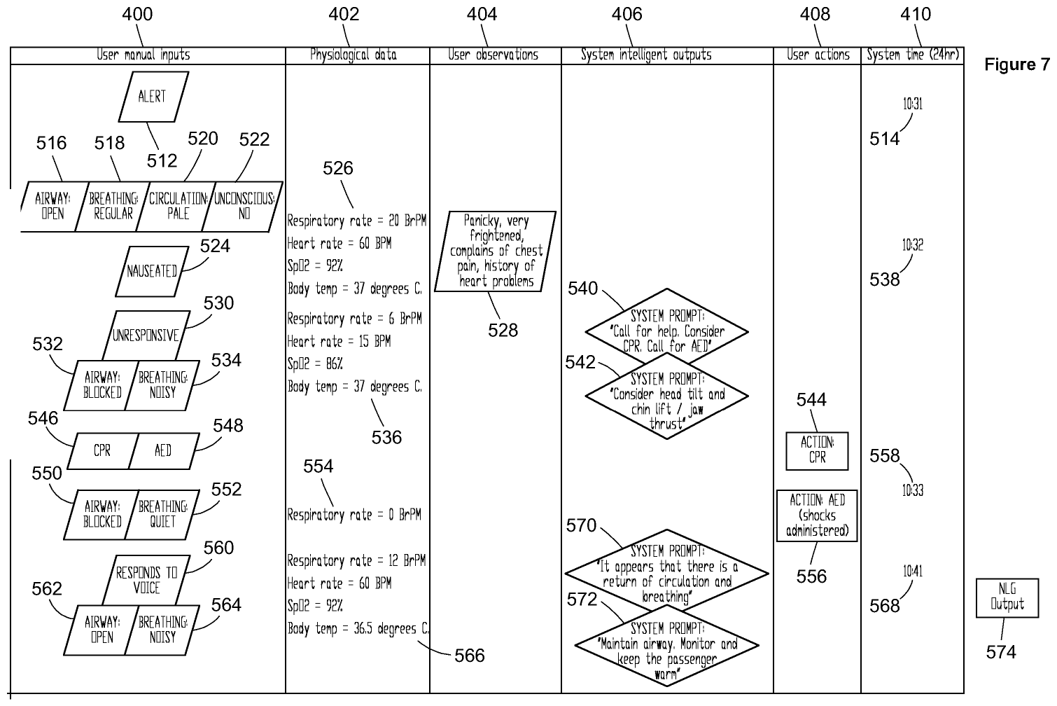

FIGS. 6 and 7 are flow charts each illustrating in overview a series of steps performed by a first response system in accordance with a respective embodiment. The first response system may be as described above with reference to FIG. 1.

Actions relating to the flow charts of FIGS. 6 and 7 are described below as being performed by a user. In some cases, more than one user may participate in the actions described. For example, different users may provide different data inputs. One user may perform first aid actions on a patient while another user inputs data to the first response system.

In the embodiment of FIG. 6, the first response system is used for choking support when a passenger is experiencing a choking event.

The flow chart of FIG. 6 comprises a plurality of columns 400, 402, 404, 406, 408, 410. For each item of the flow chart in each column, a vertical position of the item within the column is representative of a time at which the flow chart item occurred. In other embodiments, items may occur at any appropriate times and in any appropriate order.

Items in column 400 represent user manual inputs. The user manual inputs may be provided by the user in any suitable manner, for example by providing touch screen inputs on user interface 20. Data representative of each user manual input is stored by processor 12. The processor 12 may also apply a timestamp to each user manual input.

Details of user manual inputs are also provided to a ground-based medical provider using dashboard 60.

Items in column 402 represent physiological data inputs. The physiological data inputs comprise measured values for a plurality of vital sign parameters. In the embodiment of FIG. 6, the vital sign parameters are respiratory rate, heart rate, SpO2 and body temperature. In other embodiments, values for any suitable vital sign parameters may be measured and recorded.

At least some of the physiological data inputs are obtained by automatic processing of sensor inputs from the wireless sensors 40. In other embodiments, the physiological data inputs may be obtained by manual measurement.

Values for vital sign parameters may be displayed on a display screen, for example user interface 20. Values for vital sign parameters may be stored, for example in data store 14. The processor 12 or one or more of the wireless sensors 40 may apply a timestamp to values for the vital sign parameters.

Values for vital sign parameters are also displayed to the ground-based medical provider using dashboard 60. A traffic light format is used to highlight to the ground-based medical provider values for vital sign parameters that are higher or lower than a normal range. In other embodiments, any suitable method of highlighting may be used, or no method of highlighting may be used.

Items in column 404 are representative of user observations. In the present embodiment, the user observations are input by the user via the user interface 20 as free text, for example by inputting text into free text box 222 as shown in FIG. 4. In other embodiments, any suitable method of inputting user observations may be used. For example, user observations may be input as a voice input. Data representative of each user observation is stored by processor 12. The processor 12 may also apply a timestamp to each user observation.

The free text input is also provided to the ground-based medical provider using dashboard 60.

Items in column 406 are representative of intelligent outputs by the first response system. In the embodiment of FIG. 6, the intelligent outputs are system prompts which prompt a user to perform first response actions. The system prompts may be displayed on a screen, for example user interface 20. Additionally or alternatively, the system prompts may be supplied to the user in any suitable way, for example by a voice output or system chat functionality to the ground based medical provider

Items in column 408 are representative of actions taken by the user.

Items in column 410 are representative of a system time, shown in 24-hour notation.

At stage 412, a user performs a manual input to provide an indication that the patient is ALERT. The manual input may comprise touching a display element to provide an AVPU input as described above with reference to stage 74 of FIG. 2.

At stage 414, a system time of 10:31 is recorded. The system time is associated with the user input of stage 412.

At stage 416, the user provides an observation regarding the condition of the patient, for example by entering a free text input in text box 222 on a screen as described above with reference to FIG. 4. The user observation comprises the following text: ‘Unable to speak, difficulty breathing. Further information supplied—history of food blocking’.

At stage 418, the user performs a manual input of AIRWAY:BLOCKED, for example by touching display item 214 of the screen of FIG. 4 to set or toggle a value for airway of ‘Blocked’. At stage 420, the user performs a manual input of BREATHING: IRREGULAR, for example by touching display element 216 of the screen of FIG. 4 to set or toggle a value for breathing of ‘Irregular’.

At stage 422, a system time of 10:31 is recorded. The processor 12 may associate the system time of 10:31 with the user manual input of stage 418 and/or 420.

At stage 424, values for vital sign parameters comprising respiratory rate, heart rate, SpO2 and body temperature are obtained by wireless sensors 40 and are displayed on user interface 20. The values for the vital sign parameters are saved by processor 12, and may be timestamped by processor 12.

In the example of FIG. 6, the respiratory rate at stage 424 is 11 BrPM. Heart rate is 120 BPM. SpO2 is 94%. Body temperature is 37 degrees C.

The values for the vital sign parameters are displayed on user interface 20. The values for the vital sign parameters are also displayed on the dashboard 60, on which colour formatting is used to indicate which values for vital signs are normal (green), are of concern (amber) or are of high concern (red). Respiratory rate and heart rate are considered abnormal and so are shown in red. SpO2 and body temperature are considered normal and so are shown in green. In other embodiments, any suitable method of displaying values for vital sign parameters may be used. In further embodiments, one or more of the value for vital sign parameters may be recorded without being displayed to the user.

At stage 426, the decision support module 300 analyses at least some of the user manual inputs of stages 412, 418, 420; the user observation of stage 416; and the vital sign parameter values of stage 424 and outputs a system prompt comprising text to convey a suggested action to the user. The system prompt of stage 426 comprises the text “Consider head tilt and chin lift/jaw thrust”. The processor 12 instructs the user interface 20 to display the system prompt.

At stage 428, the user performs an action comprising a head tilt and jaw thrust.

At stage 430, the user performs a manual input that is indicative of a HEAD TILT and JAW THRUST. For example, the user may click on display item 264 of the screen of FIG. 4 to move to a screen for inputting treatment given, and input an indication of HEAD TILT and JAW THRUST on the screen for inputting treatment given.

At stage 432, a system time of 10:32 is recorded. The processor 12 may associate the system time of 10:32 with the user manual input at stage 430.

At stage 434, values for vital sign parameters comprising respiratory rate, heart rate, SpO2 and body temperature are obtained by wireless sensors 40 and are displayed on user interface 20. The values for the vital sign parameters are saved by processor 12, and may be timestamped by processor 12.

The respiratory rate at stage 434 is 4 BrPM. Heart rate is 130 BPM. SpO2 is 88%. Body temperature is 37.5 degrees C. Values for the vital sign parameters are also displayed to the ground-based medical provider via the dashboard 60 using the traffic light format. At stage 434, respiratory rate, heart rate, SpO2 and body temperature are all considered abnormal and so are shown in red.

At stage 436, the user provides an observation comprising the following text: ‘Ongoing difficulty with breathing. Possible obstruction’.

At stage 438, the decision support module 300 analyses at least some of preceding manual inputs, user observations and vital sign parameter values and outputs a system prompt comprising text to convey a suggested action to the user. The system prompt of stage 438 comprises the text “Consider back blows, and if unsuccessful abdominal thrusts”. The processor 12 instructs the user interface 20 to display the system prompt.

At stage 440, the user performs an action comprising 5 back blows.

At stage 442, the user performs a manual input that is indicative of 5 BACK BLOWS. For example, the user may click on display item 264 of the screen of FIG. 4 to move to a screen for inputting treatment given, and input an indication of 5 BACK BLOWS on the screen for inputting treatment given.

At stage 444, a system time of 10:34 is recorded. The processor 12 may associate the system time of 10:34 with the user manual input at stage 442.

At stage 446, values for vital sign parameters comprising respiratory rate, heart rate, SpO2 and body temperature are obtained by wireless sensors 40 and are displayed on user interface 20. The values for the vital sign parameters are saved by processor 12, and may be timestamped by processor 12.

The respiratory rate at stage 446 is 20 BrPM. Heart rate is 90 BPM. SpO2 is 94%. Body temperature is 37.5 degrees C. Values for the vital sign parameters are also displayed to the ground-based medical provider via the dashboard 60 using the traffic light format. At stage 446, respiratory rate, heart rate, SpO2 and body temperature are all considered normal and so are shown in green.

At stage 446, the user provides an observation comprising the following text: ‘Back blows successful. Obstruction cleared. Breathing improved. Patient able to speak’.

At stage 450, the user performs a manual input of AIRWAY: OPEN, for example by touching display item 214 of the screen of FIG. 4 to toggle a value for airway from ‘Blocked’ to ‘Clear’. At stage 452, the user performs a manual input of BREATHING: REGULAR, for example by touching display element 216 of the screen of FIG. 4 to toggle a value for breathing from ‘Irregular’ to ‘Regular’.

At stage 454, the processor 12 instructs the NLG module 16 to generate an NLG output. The NLG module 16 generates the NLG output by processing data including at least some of the user manual inputs, user observations, vital sign parameters and recorded system times that were obtained in the preceding stages of FIG. 6. In the example of FIG. 6, the NLG output comprises the following text:
‘The patient first presented as ALERT (10:31). Their airway was BLOCKED and breathing IRREGULAR (10:31). Initially, respiratory rate was LOW (11 BrPM) and heart rate was HIGH (120 BPM). HEAD TILT AND JAW THRUST was actioned at 10:32, following which all vital signs deteriorated. All vital signs returned to normal range following BACK BLOWS (10:34).’

In the embodiment of FIG. 7, the first response system is used in a scenario in which a patient, for example a passenger on an aircraft, is experiencing a cardiac arrest.

The flow chart of FIG. 7 comprises a plurality of columns 400, 402, 404, 406, 408, 410 as described above with reference to FIG. 6.

At stage 512 of the flow chart of FIG. 7, a user performs a manual input to provide an indication that the patient is ALERT. The manual input may comprise touching a display element to provide an AVPU input as described above with reference to stage 74 of FIG. 2.

At stage 514, a system time of 10:31 is recorded. The processor 12 may associate the system time of 10:31 with the user input of stage 512.

At stage 516, the user performs a manual input of AIRWAY: OPEN, for example by touching display item 214 of the screen of FIG. 4 to set or toggle a value for airway of ‘Clear’. At stage 420, the user performs a manual input of BREATHING: REGULAR for example by touching display element 216 of the screen of FIG. 4 to set or toggle a value for breathing of ‘Regular’. At stage 520, the user performs a manual input of CIRCULATION: PALE, for example by touching display element 218 of FIG. 4 to set or toggle a value for circulation of ‘Pale’. At stage 522, the user performs a manual input of UNCONSCIOUS: NO, for example by touching display element 254 of FIG. 4 to set or toggle a value for unconscious to ‘no’.

At stage 524, the user performs a manual input of NAUSEATED, for example by touching display element ‘240’ of the screen of FIG. 4 to set or toggle a value for whether the patient is nauseated.

At stage 526, values for vital sign parameters comprising respiratory rate, heart rate, SpO2 and body temperature are obtained by wireless sensors 40 and are displayed on user interface 20. The values for the vital sign parameters are saved by processor 12, and may be timestamped by processor 12.

The respiratory rate at stage 526 is 20 BrPM. Heart rate is 60 BPM. SpO2 is 92%. Body temperature is 37 degrees C. Values for the vital sign parameters are also displayed to the ground-based medical provider via the dashboard 60 using the traffic light format. The value for SpO2 is considered abnormal and so is displayed in red. Values for respiratory rate, heart rate, and body temperature are considered normal and so are shown in green. It is noted that oxygen saturations at altitude may be up to 5% lower than on the ground so may typically be used mainly for monitoring.

At stage 528, the user provides an observation comprising the following text: “Panicky, very frightened, complaints of chest pain, history of heart problems.”

At stage 530, the user performs a manual input of UNRESPONSIVE, for example by touching display item 212 of the screen of FIG. 4 to set or toggle a value for whether the patient responds to voice. At stage 532, the user performs a manual input of AIRWAY: BLOCKED, for example by touching display item 214 of the screen of FIG. 4 to toggle a value for airway from ‘Clear’ to ‘Blocked’. At stage 534, the user performs a manual input of BREATHING:NOISY, for example by touching display item 216 of the screen of FIG. 4 to toggle a value for breathing from ‘Irregular’ to ‘Noisy’.

At stage 536, values for vital sign parameters comprising respiratory rate, heart rate, SpO2 and body temperature are obtained by wireless sensors 40 and are displayed on user interface 20. The values for the vital sign parameters are saved by processor 12, and may be timestamped by processor 12. The respiratory rate is 6 BrPM. Heart rate is 15 BPM. SpO2 is 86%. Body temperature is 37 degrees C. Values for the vital sign parameters are also displayed to the ground-based medical provider via the dashboard 60 using the traffic light format. Values for respiratory rate, heart rate, and body temperature are all considered abnormal and so are shown in red.

At stage 538, a system time of 10:32 is recorded. The processor 12 may associate the system time with one or more of the user inputs of stages 530, 532 and 534 and/or with the values for vital sign parameters at stage 536.

At stage 540, the decision support module 300 analyses at least some of the preceding manual inputs, user observation and vital sign parameter values and outputs a system prompt comprising text to convey a suggested action to the user. The system prompt of stage 540 comprises the text “Call for help. Consider CPR. Call for AED”. The processor 12 instructs the user interface 20 to display the system prompt.

At stage 542, the decision support module 300 analyses at least some of the preceding manual inputs, user observation and vital sign parameter values and outputs a system prompt comprising text to convey a suggested action to the user. The system prompt of stage 542 comprises the text ‘Consider head tilt and chin lift/jaw thrust’. The processor 12 instructs the user interface 20 to display the system prompt.

At stage 544, the user performs an action of performing CPR.

At stage 546, the user performs a manual input that is indicative of CPR and at stage 548 the user performs a manual input that is indicative of AED. For example, the user may click on display item 264 of FIG. 4 to move to a screen for inputting treatment given, and input the indications of CPR and AED on the screen for inputting treatment given.

At stage 550, the user performs a manual input of AIRWAY: BLOCKED, for example by confirming a value of display element 214 of FIG. 4. At stage 552, the user performs a manual input of BREATHING: QUIET, for example by touching display element 216 to toggle a value for breathing from ‘Noisy’ to ‘Quiet’.

At stage 554, a wireless sensor 40 records a value for respiratory rate of 0 BrPM. The respiratory rate value is displayed on user interface 20. The respiratory rate value is saved by processor 12, and may be timestamped by processor 12. The respiratory rate value is also displayed to the ground-based medical provider using dashboard 60.

At stage 556, the user performs an action of administering shocks using the AED.

At stage 558, a system time of 10:33 is recorded. The processor may associate the system time of 10:33 with one or more of the user inputs of stages 546, 548, 550 and 552 and/or the respiratory rate value of stage 554.

At stage 560, the user performs a manual input of RESPONDS TO VOICE, for example by touching display element 212 of FIG. 4 to toggle a value for whether the patient responds to voice. At stage 562, the user performs a manual input of AIRWAY:OPEN, for example by touching display element 214 of FIG. 4 to toggle a value for airway from ‘Blocked’ to ‘Clear’. At stage 564, the user performs a manual input of BREATHING: NOISY, for example by touching display element 216 of FIG. 4 to toggle a value for breathing from ‘Quiet’ to ‘Noisy’.

At stage 566, values for vital sign parameters comprising respiratory rate, heart rate, SpO2 and body temperature are obtained by wireless sensors 40 and are displayed on user interface 20. The values for the vital sign parameters are saved by processor 12, and may be timestamped by processor 12. The respiratory rate is 12 BrPM. Heart rate is 60 BPM. SpO2 is 92%. Body temperature is 36.5 degrees C. Values for the vital sign parameters are also displayed to the ground-based medical provider via the dashboard 60 using the traffic light format. The value for SpO2 is considered abnormal and so is displayed in red. The values for respiratory rate, heart rate, and body temperature are considered normal and so are shown in green.

At stage 568, a system time of 10:41 is recorded. The processor may associate the system time of 10:41 with one or more of the user inputs of stages 560, 562, and 564 and/or any of the values for vital sign parameters of stage 566.

At stage 570, the decision support module 300 analyses at least some of the preceding manual inputs, user observation and vital sign parameter values and outputs a system prompt. The system prompt of stage 570 comprises the text “It appears that there is a return of circulation and breathing’. The processor 12 instructs the user interface 20 to display the system prompt.

At stage 572, the decision support module 300 analyses at least some of the preceding manual inputs, user observation and vital sign parameter values and outputs a system prompt the processor 12 instructs the user interface 20 to display a system prompt. The system prompt comprises the text ‘Maintain airway. Monitor and keep the passenger warm’. The processor 12 instructs the user interface 20 to display the system prompt.

At stage 574, the processor 12 instructs the NLG module 16 to generate an NLG output. The NLG module 16 generates the NLG output by processing data including at least some of the user manual inputs, user observations, vital sign parameters and recorded system times that were obtained in the preceding stages of FIG. 7. The NLG output comprises the following text:
‘The patient first presented as ALERT, with an OPEN AIRWAY, REGULAR BREATHING and PALE (all 10:31). They had NOT been UNCONSCIOUS. They were NAUSEOUS complaining of CHEST pain, and had a history of HEART PROBLEMS. At 10:32 they became UNRESPONSIVE. Their airway was BLOCKED and their breathing NOISY. Around the same time their respiratory rate, heart rate and blood oxygen saturation deteriorated significantly. CPR commenced, and an AED was used to deliver shocks. By 10:41 all physiology had improved to normal parameters, the airway was OPEN with NOISY breathing.’

The interactive steps outlined above, for example with reference to FIGS. 6 and 7, may prompt the user to take one or more appropriate actions. Patient status may be highlighted to the responder. This type of interactivity, in combination with multiple sensor data streams, and NLG report, may provide a system with significant differences over a basic monitoring system which may purely capture and/or display sensor data. A combined approach including user prompts and patient status may further assist first responders.

In embodiments described above with reference to FIGS. 6 and 7, information is displayed on both the user interface 20 and the dashboard 60. In other embodiments, information may be displayed on the user interface 20 without being transmitted to a dashboard 60. In further embodiments, some information may be displayed on the dashboard 60 without being displayed on the user interface 20. Different formatting may be used for the dashboard 60 when compared with the user interface 20.

A skilled person will appreciate that variations of the enclosed arrangement are possible without departing from the invention. Accordingly, the above description of the specific embodiment is made by way of example only and not for the purposes of limitations. It will be clear to the skilled person that minor modifications may be made without significant changes to the operation described.

1. A portable apparatus for use by first responders, the apparatus comprising:
at least one receiver configured to receive input from at least one medical sensor, wherein the input comprises or is representative of sensor data relating to a human subject;
a display device configured to display first response guidance to a user;
a user input device configured to receive input from the user; and
processing circuitry configured to:
process the input from the at least one medical sensor and the input from the user;
select the first response guidance to be displayed to the user via the display device, the selecting of the first response guidance comprising:
estimating a status of the human subject based on the input from the at least one medical sensor, and
selecting at least one item of first response guidance from a plurality of stored items of first response guidance, wherein the selecting is based on the estimated status;
wherein the first response guidance comprises at least one action to be performed by the user in relation to the human subject.

2. The apparatus according to claim 1, wherein the processing circuitry is further configured to automatically generate and output a medical handover report based on the input from the at least one medical sensor and the input from the user, wherein the medical handover report summarises medical information relating to the human subject.

3. The apparatus according to claim 1, wherein the apparatus is an aviation apparatus for use by cabin crew.

4. The apparatus according to claim 1 wherein the receiver, display device, user input device and processing circuitry are housed within a single housing.

5. The apparatus according to claim 1 wherein the at least one medical sensor comprises at least one wireless sensor, and wherein the at least one receiver is configured to receive the input from the at least one wireless sensor via a wireless connection, optionally wherein the wireless connection comprises a Bluetooth® connection.

6. The apparatus according to claim 1 wherein the at least one medical sensor comprises at least one of an electrocardiography (ECG) sensor, a pulse oximeter, a heart rate monitor, a blood pressure sensor, or a temperature sensor.

7. The apparatus according to claim 1, wherein at least one of:
a) the first response guidance is simplified guidance for use by first responders who are not medical professionals; or
b) the first response guidance is generated in accordance with a standard first response protocol.

8. (canceled)

9. The apparatus according to claim 1, wherein the estimating of the status of the human subject is further based on the input from the user.

10. The apparatus according to claim 1, wherein the least one action to be performed by the user in relation to the human subject comprises at least one of:
a) administering a treatment to the human subject, optionally wherein the treatment comprises oxygen;
b) inputting information relating to at least one sign or symptom of the human subject; or
c) obtaining a manual measurement of a parameter relating to the human subject and inputting the manual measurement via the user input device.

11. The apparatus according to claim 1, wherein the processing circuitry is further configured to generate an alarm in dependence on the estimated status and/or the input from the at least one medical sensor and/or the input from the user.

12. The apparatus according to claim 1,
wherein at least one of:
a) the generating of the medical handover report comprises using natural language generation to generate a natural language description of information relating to the human subject and actions performed in relation to the human subject;
b) the generating of the medical handover report comprises collating the input from the at least one sensor and the input from the user; and selecting or prioritising information from the collated input that is most relevant to the medical handover report; or
c) the handover report is structured in accordance with a standard medical format, for example an ATMIST format.

13-14. (canceled)

15. The apparatus according to claim 1, wherein the processing circuitry is configured to automatically timestamp the input from the at least one medical sensor and/or the input from the user to obtain timestamp data, and to use the timestamp data in the generating of the handover report.

16. The apparatus according to claim 1, wherein the processing circuitry is further configured to automatically generate and output a medical incident report based on the input from the at least one medical sensor and the input from the user, for example wherein the medical incident report comprises an airline incident report.

17. The apparatus according to claim 1, further configured to transmit data from the apparatus to an aircraft communications system, for example via in-flight Wi-Fi.

18. The apparatus according to claim 17, wherein at least one of:
a) the transmitting of data is performed in real time; or
b) the transmitting of data comprises receiving text input from the user of the apparatus and transmitting the text input and the processing circuitry is further configured to receive and process a text reply, thereby providing text chat functionality.

19. (canceled)

20. The apparatus according to claim 1, wherein the processing circuitry is further configured to apply conditional formatting to the user interface to highlight specific display items of the user interface in dependence on the estimated status of the human subject.

21. The apparatus according to claim 1, wherein at least one of:
a) the user interface is configured to receive input from the user via a one-touch input method; or
b) the processing circuitry is configured to record and timestamp one-touch input from the user.

22. (canceled)

23. The apparatus according to claim 1 further comprising the at least one medical sensor.

24. A method comprising:
receiving, by at least one receiver, input from at least one medical sensor, wherein the input comprises or is representative of sensor data relating to a human subject;
displaying, by a user interface, first response guidance to a user;
receiving, by the user interface, input from the user;
processing, by processing circuitry, the input from the at least one medical sensor and the input from the user; and
selecting, by the processing circuitry, the first response guidance to be displayed to the user via the user interface, the selecting of the first response guidance comprising:
estimating a status of the human subject based on the input from the at least one medical sensor, and
selecting at least one item of first response guidance from a plurality of stored items of first response guidance, wherein the selecting is based on the estimated status;
wherein the first response guidance comprises at least one action to be performed by the user in relation to the human subject.

25. An article of manufacture including a non-transitory, tangible computer readable storage medium having instructions stored thereon that, in response to execution by a processor, cause the processor to perform operations comprising:
receiving, by at least one receiver, input from at least one medical sensor, wherein the input comprises or is representative of sensor data relating to a human subject;
displaying, by a user interface, first response guidance to a user;
receiving, by the user interface, input from the user;
processing, by the processor, the input from the at least one medical sensor and the input from the user; and
selecting, by the processor, the first response guidance to be displayed to the user via the user interface, the selecting of the first response guidance comprising:
estimating a status of the human subject based on the input from the at least one medical sensor, and
selecting at least one item of first response guidance from a plurality of stored items of first response guidance, wherein the selecting is based on the estimated status;
wherein the first response guidance comprises at least one action to be performed by the user in relation to the human subject.
